(19) United States
(12) Patent Application Publication
(10) Pub. No.: US 2009/0285913 A1
(43) Pub. Date: Nov. 19, 2009

(54) Trachelospermi Caulis Extract Composition for the Treatment and Prevention of Inflammatory Diseases

(76) Inventors: Jeong Min Lee, Gyeonggi-do (KR); Chul Gyu Ha, Gyeonggi-do (KR); Mu Hong Lee, Gyeonggi-do (KR); Seung Ha Lee, Gyeonggi-do (KR); Jae Yoon Leem, Jeollabuk-do (KR); Sung Hoon Jun, Chungcheongbuk-do (KR); Wahn Soo Choi, Seoul (KR); Jee Hun Park, Seoul (KR)

Correspondence Address: FROMMER LAWRENCE & HAUG, 745 FIFTH AVENUE- 10TH FL., NEW YORK, NY, 10151, US

(21) Appl. No.: 12/307,091

(22) PCT Filed: Jul. 3, 2007
(86) PCT No.: PCT/KR07/03225

§ 371 Date: Dec. 30, 2008

(30) Foreign Application Priority Data
Jul. 3, 2006 (KR) 10-2006-0062057
Jan. 19, 2007 (KR) 10-2007-0006239

(51) Int. Cl. A61K 36/24; A61P 29/00

(52) U.S. Cl. 424/725

(57) ABSTRACT

The present invention relates to a pharmaceutical composition for the prevention and treatment of inflammatory diseases comprising The present invention relates to a pharmaceutical composition for the prevention and treatment of inflammatory diseases comprising Trachelospermi caulis extract as an active ingredient. More particularly, the pharmaceutical composition of the present invention is characterized in that it is standardized and formulated so that the arctiin is contained in the Trachelospermi caulis extract within a predetermined range to exhibit excellent inhibitory effects on pains, acute inflammation, acute edema, production of iNOS, and TNF-Yá, activation of the enzymes of MAP kinases and NFYêB, thus effective in the prevention and treatment of inflammatory diseases such as arthritis.

### BACKGROUND OF THE INVENTION

1. Field of the Invention

The present invention relates to a pharmaceutical composition for the prevention and treatment of inflammatory diseases comprising Trachelospermi caulis extract as an active ingredient. More particularly, the pharmaceutical composition of the present invention is characterized in that it is standardized and formulated so that the arctiin is contained in the Trachelospermi caulis extract within a predetermined range to exhibit excellent inhibitory effects on pains, acute inflammation, acute edema, production of inducible nitric oxide synthase (iNOS), and TNF-α, activation of MAP kinases (p-p38, p-Erk, p-JNK) and NFκB, thus effective in the prevention and treatment of inflammatory diseases such as arthritis.

2. Background Art

Arthritis is a collective term for a disease associated with inflammatory changes which occur within a joint region due to bacterial infection or external injuries. Arthritis is largely classified into two: acute arthritis and chronic arthritis.

Acute arthritis is further divided as follows. (1) Serous arthritis—generally caused by external injuries, but may occur due to unknown reasons. It generally occurs in one joint. (2) Serofibrinous arthritis—this occurs with the acute rheumatoid arthritis, and a turbid effusion is gathered in the articular cavity. This may cause dyscinesia even after inflammation is deteriorated due to the generation of pseudomembrane. (3) Supprative arthritis—multiple arthritis occurs in the open wounds of a joint or contagious diseases such as gonorrhea, typhoid, scarlatinal, and septicemic. Young infants of 1-2 months old may develop abarticulation due to the uncurable severe damage on bones. Adults often develop periosteomyelitis which causes rupture and allows the pus to be flowed into joints, so called secondary supprative arthritis.

Chronic arthritis can be further divided as follows.

(1) Specific type of inflammation—generally refers to a gouty arthritis caused by tuberculous arthritis or syphilitic arthritis or metabolic disorder in uric acid commonly occurring in middle aged men.

(2) Multiple arthritis—chronic rheumatoid arthritis is most common. This may be transited from the acute serous arthritis, may occur as a polyarthritis in the course of pneumonia, syphilis, and gonorrhea, or may be a kind of septicemia. In addition, Still's disease also belongs to this category.

(3) Arthritis deformans—generally caused by degenerative aging process or external injuries.

(4) Hemophiliac arthritis—caused by bleeding in the joints in a hemophiliac patient.

Many pharmaceutical drugs have been developed in order to be used for the treatment of the above-mentioned various kinds of inflammatory diseases represented by arthritis.

Trachelospermi caulis is also called as Trachelospermum asiaticum var. intermedium Nakai, and Lonicera sempervirens which belongs to family of Apocyanaceae. It grows as high as 5 m. Its petals are branched deep inside into five. White flowers of a pinwheel shape bloom in May-June and emits a good scent. It will bear fruits around September-November, where the two long fruits remind of a Chinese character representing a person, and they often form a round circle showing the shape of a bracelet. In fact, Trachelospermi caulis grows in the seashore, hillside or a barren tract in the Southern part of Korea, more specifically, on the rocks, walls, or grows creepers onto other trees or plants. In areas where Trachelospermi caulis is abundant, it is possible that other kinds of grasses may not grow but only the place may be clothed with Trachelospermi caulis. According to the ‘Dictionary of Oriental Medicine’ published by North Korea, Trachelospermi caulis is the one which dried the stems and leaves of Trachelospermum asiaticum var. intermedium Nakai. It has a bitter taste and is cold in its property from the viewpoint of oriental medicine. It acts on heart meridian, liver meridian, and renal meridian. It also eliminates wind-dampness blended as a pathogenic factor and promotes smooth interconnection of meridian pathways. Besides, it is useful to treat paralytic syndrome, cramps of limbs, lumbago, arthritic pains, tonsillitis, and rashes. About 5-10 g of Trachelospermi caulis is decocted and administered orally. Further, it is used to treat limbs paralysis due to wind-dampness blended as a pathogenic factor, muscular paralysis, difficulty in bending and stretching bodies, by adding dried root bark of Acanthopanax sessiliflorus (Rupr. et Max.) and dried root of Achyranthes bidentata Bl. It also cools blood and is thus used to treat a sore throat, swellings via oral administration after decocting it using water.

The fruits of Trachelospermi caulis use for medicinal purposes. The ripen fruits are collected and 6-12 g is decocted to be administered orally to treat bone and muscle aches. Leaves and stems are mostly used. They can be collected any time regardless of seasons, dried in the sunlight, minced into small pieces and then used. According to Chinese Honam medicine paper, it is recommended treating people with bone and muscle aches by oral administration of Trachelospermi caulis prepared by soaking 37-74 g of Trachelospermi caulis in an alcoholic beverage. According to Kangseo herbal medicine, it is recommended treating people suffering from arthritis by oral administration of a liquid herbal medicine prepared by decocting a mixture comprising 37 g of Trachelospermum asiaticum var. intermedium Nakai, 37 g of Acanthopanacis Cortex, and 18.5 g of hyssop using water along with white liquor. It is preferable to prepare a liquid herbal medicine by decocting 8-12 g of dried Trachelospermi caulis in 200 mL of water until the entire volume is decreased to one third. In addition, it can be administered after soaking it in an alcoholic beverage or in the form of powder. For external use, it is applied to the wounded area after pulverizing it into powder or pasting or rinsing with the juice extract produced by pounding the fresh leaves of Trachelospermi caulis.

Trachelospermi caulis is Apocyanaceae used to treat lumbago, strengthen bones and muscles, and make joints smooth. Trachelospermi caulis is known to contain as components tracheloside, arctiin, matairesinoside, arctigenin, notracheloside, etc.

Trachelospermi caulis has been described as simple prescription in the old herbal encyclopedia such as Donguibogam, Hyangyakjipsungbang, Gwangjebigeup. However, these prescriptions were merely mentioned briefly on the ways of identification by external appearance, efficacies from the oriental herbal medicine, and the method of preparing decoction. That is, there have been no teachings on the active ingredients of Trachelospermi caulis.

In addition, the content of a biomarker of Trachelospermi caulis differs greatly depending on the time of collection and place of production and thus it is very difficult to achieve standardization of Trachelospermi caulis extract. Further, active fractions having excellent therapeutic activities against inflammation, pains, improvement of blood circulation, arthritis, are hardly reproducible thus impeding its commercialization.

The information disclosed in this Background of the Invention section is only for enhancement of understanding of the background of the invention and should not be taken as an acknowledgement or any form of suggestion that this information forms the prior art that is already known to a person skilled in the art.

### SUMMARY OF THE INVENTION

The present invention has been made in an effort to achieve standardization of Trachelospermi caulis extract by means of controlling its components and their respective contents which have excellent inhibitory activities against inflammatory symptoms such as inflammations and pains, expression of enzymes such as p-p38 MAP and p-NF κB, acute inflammation, acute edema, etc.

As a result, the inventors of the present invention discovered that Trachelospermi caulis extract comprising a certain amount of active fractions having excellent inhibitory activities against general inflammatory symptoms can be easily and reproducibly obtained thus enabling its commercialization.

In one aspect, the present invention provides Trachelospermi caulis extract effective in the treatment and prevention of inflammatory diseases.

In another aspect, the present invention provides Trachelospermi caulis extract which comprises a predetermined amount of arctiin, a biomarker for active fractions having inhibitory activities against general inflammatory diseases.

### BRIEF DESCRIPTION OF THE DRAWINGS

The above and other features of the present invention will be described with reference to certain exemplary embodiments thereof illustrated the attached drawings in which:

FIG. 1 is an HPLC chromatogram of the Trachelospermi caulis extract prepared according to an embodiment of the present invention;

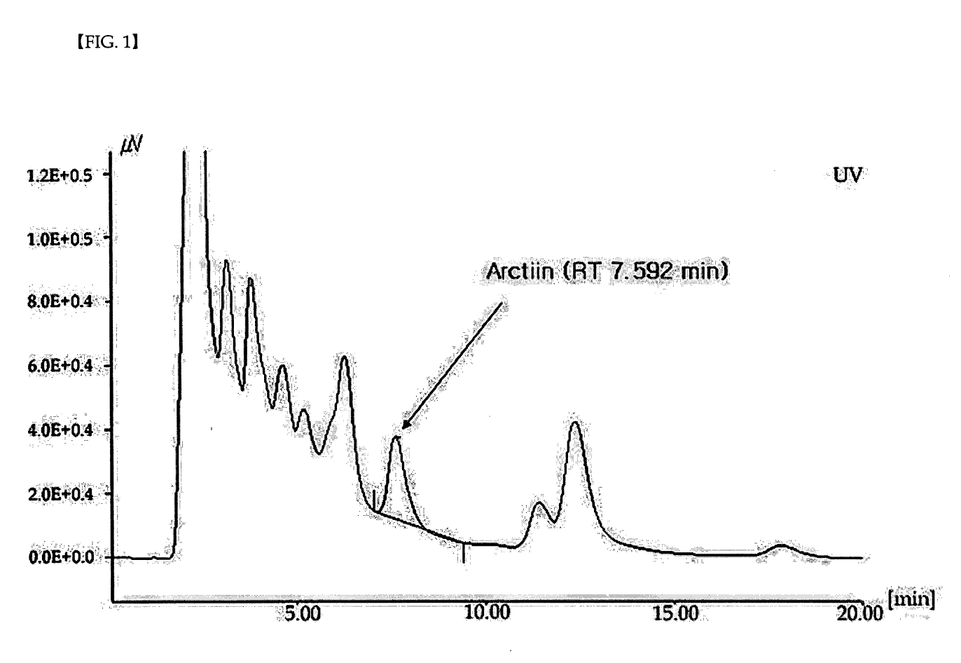

FIGS. 2A and 2B show the effect of Trachelospermi caulis on LPS-induced iNOS expression, NO production in murine macrophage RAW 264.7 cells (in FIG. 2A, the cells were treated with Trachelospermi caulis (TC) or Joins in the indicated doses 30 min before incubating them with 1 ug mL⁻¹ LPS for 4 h, and the levels of iNOS protein were determined using Western blot analysis; and in FIG. 2B, the concentration of nitrite in the culture media was monitored through the Griess reaction, one-hundred microliters of each supernatant was mixed with the same volume of Griess reagent (1% sulfanilamide in 5% phosphoric acid, and 0.1% naphthylethylenediamine dihydrochloride in water), and the resulting mixture was then incubated at room temperature for 10 min. Its absorbance at 540 nm was then measured in a microplate reader); and

FIGS. 3A and 3B show the effect of Trachelospermi caulis on the activating phosphorylation of p-38 and NF-kB in murine macrophage RAW 264.7 cell (in FIG. 3A, the cells were treated with T. caulis (TC) or Joins in the indicate dose 30 min before incubating them with 1 μg mL⁻¹ LPS for 4 h and activating phosphorylation of p38. ERK and JNK were measured using specific antibodies by Western blot analysis as described in the methods section; and in FIG. 3B, the cells were treated with TC or Joins in the indicated dose 30 min before incubating them with 1 μg mL⁻¹ LPS for 10 min, and the activating phosphorylation of NF-kB was measured by Western blot analysis as described previously, and the data are presented as the means ±s.e.m. from three independent experiments. *p<0.05, p<0.01 compared with the control).

### DETAILED DESCRIPTION

In an embodiment of the present invention, there is provided a pharmaceutical composition comprising Trachelospermi caulis extract as an active ingredient useful for the prevention and treatment of inflammatory diseases.

In another embodiment of the present invention, there is provided a pharmaceutical composition comprising arctiin, a biomarker for the Trachelospermi caulis extract, in the amount of 3.0-6.0 wt %, useful for the prevention and treatment of inflammatory diseases.

The present invention is described in more detail as set forth hereunder.

The present invention relates to Trachelospermi caulis extract which is standardized to contain a suitable amount of arctiin, its active ingredient, to such a level to sufficiently express inhibitory activities against pains, expression of cytokines of p-p38 MAP and p-NF κB, acute inflammation, acute edema, thus being used in manufacturing a pharmaceutical composition useful for the prevention and treatment of inflammatory diseases such as arthritis.

The pharmaceutical composition for the prevention and treatment of inflammatory diseases prepared according to an embodiment of the present invention comprises Trachelospermi caulis extract as an active ingredient. Preferably, the Trachelospermi caulis extract contains arctiin as a biomarker in the amount of 3.0-6.0 wt %. p-p38 MAP and p-NF κB, acute inflammation, acute edema, etc.

In general, the contents of physiologically active substances vary greatly depending on the habitat, harvest time, period and conditions of storage. Therefore, it is preferable to limit the content of certain active ingredients of a herbal drug in determining the composition of the herbal drug instead of limiting weight ratio of ingredients. In the present invention, therefore, arctiin was selected as a biomarker to obtain the Trachelospermi caulis extract. The molecular formula of the Trachelospermi caulis extract is C₂₇H₃₄O₁₁.

The Trachelospermi caulis extract of the present invention used as a therapeutic agent for the treatment of inflammatory diseases can be the extract itself or a certain active fraction obtained by extraction of Trachelospermi caulis. Here, the mixing ratio is preferably determined by the content of the biomarker, arctiin, regardless of the content of Trachelospermi caulis.

That is, for a therapeutic agent for the treatment of inflammatory disease according to the present invention, the intended therapeutic effect could be obtained when the content of arctiin, as a biomarker, was contained in the amount of 3.0-6.0 wt %. For example, if the content of arctiin is less than 3.0 wt %, the therapeutic effect for the treatment of arthritis, for example, is greatly deteriorated. There is no specific upper limit in the content of arctiin. However, the arctiin content exceeding the above range will not increase the intended therapeutic effect, and it is not also desirable in its technical and economical aspects. Therefore, it is preferable that the arctiin content be contained about 4.0 wt %.

Arctiin, the biomarker of the present invention, is an essential component of the therapeutic agent for the treatment of inflammatory diseases, and it can help a given therapeutic drug to exhibit an excellent drug efficacy by exerting a synergistic effect when it is contained a certain amount. Further, other ingredients, not only the above active ingredient but other ingredients of the therapeutic agent may also be involved in exerting the excellent therapeutic effect for the treatment of inflammatory diseases.

A method of preparing the Trachelospermi caulis extract of the present invention useful in the treatment of inflammatory diseases comprises:

(a) extracting Trachelospermi caulis with 5-8 times of water, alcohol, or an aqueous solution relative to the weight of the Trachelospermi caulis, followed by cooling and filtration;

(b) performing layer separation of the filtrate obtained in step (a) using an aqueous solution and then concentrating it at 60-80° C. under reduced pressure; and

(c) adding alcohol to the concentrate obtained in step (b), centrifuging the mixture at 500-1,000 rpm, concentrating the resulting filtrate under reduced pressure, followed by drying under vacuum, pulverization and sterilization.

The above method is described in more detail as follows.

The natural herb of Trachelospermi caulis is added with water, alcohol or an aqueous alcohol solution, and performs extraction 2-5 times for 2-3 hours of unit period of extraction. The resultant is slowly cooled down at room temperature, and separated from the residue by filtration via centrifugation. The resulting residue is then added with 5-8 times of water, alcohol or an aqueous alcohol solution, relative to the weight of the herbal drug, heated, reextracted, filtrated, combined with the previous filtrate and then filtrated. The reextraction by adding water, alcohol or an aqueous alcohol solution to the residue followed by filtration can increase the extraction efficiency. Here, if the amount of water, alcohol or an aqueous alcohol solution is less than 5 times it will reduce solubility of the resulting extract thereby decreasing extraction efficiency. Meanwhile, if the amount of water, alcohol or an aqueous alcohol solution exceeds 8 times it will require an increased amount of alcohol to be used and also a longer time for concentration under reduced pressure thus being uneconomical and causing handling problems.

As for the alcohol of the present invention, both aliphatic and aromatic alcohols may be used, preferably aliphatic alcohol, more preferably a C₁-C₆ low grade alcohol.

In the present invention, an additional extraction was performed after the first extraction. This is to reduce the loss of extract occurring in bulk extraction of herbal drug due to a relatively high water content of the herbal drug which cannot be avoided even with efficient filtration methods. Further, analysis of extraction efficiency showed that the reextraction enables to obtain about 80-90% of the total extract while multiple extractions of more than three extractions are shown not economical.

As mentioned above, the extracts obtained by extracting with water, alcohol or an aqueous solution, are filtrated, concentrated, and then unnecessary proteins, polysaccharides, and fatty acids contained in the filtrate as impurities are purified. In the present invention, the impurities were removed by performing layer separation for the filtrate 2-5 times by using the same amount of an aqueous alcohol solution as a solvent and obtaining a solvent fraction therefrom.

It is preferable that the above alcohol is a C₁-C₆ alcohol, and more preferably 30% ethanol. If the amount of low grade aqueous alcohol is less than that of the filtrate it would prevent the process of a smooth layer separation due to the formation of granules by unnecessary components such as fatty acids, and is also not economical because the extracted contents of active ingredients are lowered.

The extraction liquid obtained after layer separation is concentrated at 60-80° C. under reduced pressure to remove the remaining solvent. Thus obtained concentrate is added with an alcohol collected during the concentration under reduced pressure, added with the filtrate obtained after centrifugation at 500-1000 rpm and concentrated again under reduced pressure. Here, if the temperature for the concentration under reduced pressure is below 60° C. the solvent cannot be removed completely. Meanwhile, if the temperature is above 80° C. it would raise a problem in the stability of the concentrate. If the concentrate is less than 500 rpm the separation of the concentrate from alcohol becomes difficult. In contrast, if it exceeds 1000 rpm it would raise a problem in the stability of the concentrate.

Thus obtained concentrate is dried at 60-80° C. under 0.08-0.3 pa, and sterilized by passing through a 30-80 mesh sieve and the Trachelospermi caulis extract in the form of powder is finally obtained. Thus obtained the Trachelospermi caulis extract has superior therapeutic effects for the treatment of arthritis and the like and thus the herbal drug comprising the Trachelospermi caulis extract will be useful for the protection of joints.

The Trachelospermi caulis extract of the present invention can be formulated by using a conventional method into tablets, capsules, injections, etc. If the combined amount of lactose, microcrystalline cellulose, magnesium stearate, etc., used as a base material for manufacturing tablets, is used along with the Trachelospermi caulis extract in 1:1 weight ratio, the tablets manufactured thereof will have therapeutic effects for the treatment of inflammatory diseases such as arthritis, edema, etc., and an analgesic effect.

Herbal extracts themselves can be used as a therapeutic agent but they are in general combined with a carrier, a forming agent, a diluent, etc., and prepared into powder, granulates, capsules, or injections. The Trachelospermi caulis extract of the present invention has long been used as food as well as a drug. It has no special limit with regard to its dosage but it the dosage may vary depending on the rate of body absorption, body weight, age, sex, health conditions, diet of a patient, administration time, administration method, excretion rate, severeness of diseases, etc. In general, it is preferable to administer about 0.1-1000 mg of the Trachelospermi caulis extract per 1 kg of body weight.

Therefore, the composition comprising the active ingredient of the present invention should be manufactured considering its effective range, and a unit dosage preparation formulated thereof can be monitored of its administration, if necessary. Further, a specialized administration method may be used according to the decision and request of the patient or it may be administered a few times at regular intervals.

Reference will now be made in detail to the preferred embodiment of the present invention, examples of which are illustrated in the drawings attached hereinafter, wherein like reference numerals refer to like elements throughout. The embodiments are described below so as to explain the present invention by referring to the figures.

### Example 1

### Preparation of Trachelospermi caulis Extract

Trachelospermi caulis was washed with water, dried and then stirred after adding 30% ethanol, and heat-extracted twice at 2 hour unit. The resulting extract was cooled down to room temperature and performed centrifugal filtration to remove impurities. The filtrates were combined and concentrated at 60-80° C. under reduced pressure. The concentrate was suspended in the ethanol recovered from the ethanol fraction, underwent centrifugal filtration at 1000 rpm, concentrated at 60° C. under reduced pressure, dried under the pressure of 0.08 pa, sterilized by passing through a 80 mesh sieve. The Trachelospermi caulis extract obtained as a result was shown to contain 4.0 wt % of arctiin. The Trachelospermi caulis extract was also analyzed by HPLC on the following conditions.

1) Eluent: A: 50% methanol

2) Column: C-18 COSMOSIL PACKED, 10 μm, 4.6×250 mm

3) Flow rate: 0.8 mL/min

4) Column temp.: 20° C.

5) Detector: UV 280 nm

### Experimental Example 1

### TPA-Induced Mice Ear Edema Assay

The effect of Trachelospermi caulis extract obtained in Example 1 to inhibit TPA-induced mouse ear edema was investigated. The result is shown in the following Table 1.

[Test Method 1]

Twenty-four hour fasted 7 week old ICR mice were separated into each experimental group, orally administered with Joins® (SK Pharma Co., Ltd., Korea) at a concentration of 400 mg/kg and 200 mg/kg, and Trachelospermi caulis extract at a concentration of 400 mg/kg, 200 mg/kg and 20 mg/kg, respectively. One hour after the oral administration, each mouse was treated with TPA (2.5 (g/20 μL) after dissolving it in acetone, thereby inducing edema. During the experiment, an investigator fixed the subject tightly from the rear side and a second investigator stimulated an ear of each mouse with the edema-inducing material using a micropipette. Four hours later, ear edema was observed from mice in each experimental group. Mice were sacrificed via cervical dislocation for accurate observation.

TABLE 1

| | | Edema |
|---|---|---|
| | Ear Thickness ± | Inhibitory |
| Treatment | S.D. | Rate (%) |
| Negative Control | 0.674 ± 0.020 | — |
| Positive Control (SK- Joins ®) | 0.317 ± 0.075 | 53 |
| 400 mg/kg | | |
| Positive Control (SK- Joins ®) | 0.4 ± 0.045 | 41 |
| 200 mg/kg | | |
| Trachelospermi caulis (400 mg/kg - | 0.232 ± 0.026 | 66 |
| 4.0 wt % of arctiin content) | | |
| Trachelospermi caulis (200 mg/kg - | 0.24 ± 0.024 | 63 |
| 4.0 wt % of arctiin content) | | |
| Trachelospermi caulis (20 mg/kg - | 0.588 ± 0.047 | 13 |
| 4.0 wt % of arctiin content) | | |

1. Data represent the mean of difference in ear thickness (mm) ± S.T.D. (n = 12)
2. Control: Experimental group without drug treatment after inducing edema

As shown in the above Table 1, the Trachelospermi caulis extract of the present invention showed an excellent inhibitory effect against the TPA-induced ear edema. In particular, the highest edema inhibiting rate was observed when the concentration of the Trachelospermi caulis extract used was 400 mg/kg.

### Experimental Example 2

### Arachidonic Acid-Induced Mice Ear Edema Assay

The effect of Trachelospermi caulis extract obtained in Example 1 to inhibit arachidonic acid-induced mouse ear edema was investigated. The result is shown in the following Table 2.

[Test Method 2]

Twenty-four hour fasted 7 week old ICR mice were separated into each experimental group, orally administered with Joins® (SK Pharma Co., Ltd., Korea) at a concentration of 400 mg/kg and 200 mg/kg, and Trachelospermi caulis extract at a concentration of 400 mg/kg, 200 mg/kg and 20 mg/kg, respectively. One hour after the oral administration, each mouse was treated with arachidonic acid (2 mg/20 μL) after dissolving it in acetone, thereby inducing edema. During the experiment, an investigator fixed the subject tightly from the rear side and a second investigator stimulated an ear of each mouse with the edema-inducing material using a micropipette. One hour later, ear edema was observed from mice in each experimental group. Mice were sacrificed via cervical dislocation for accurate observation.

TABLE 2

| | | Edema |
|---|---|---|
| | Ear Thickness ± | Inhibitory |
| Treatment | S.D. | Rate (%) |
| Negative Control | 0.55 ± 0.01 | — |
| Positive Control (SK- Joins ®) | 0.27 ± 0.02 | 51 |
| 400 mg/kg | | |
| Positive Control (SK- Joins ®) | 0.3 ± 0.02 | 45 |
| 200 mg/kg | | |
| Trachelospermi caulis (400 mg/kg - | 0.22 ± 0.03 | 60 |
| 4.0 wt % of arctiin content) | | |
| Trachelospermi caulis (200 mg/kg - | 0.24 ± 0.05 | 56 |
| 4.0 wt % of arctiin content) | | |
| Trachelospermi caulis (20 mg/kg - | 0.55 ± 0.02 | 0 |
| 4.0 wt % of arctiin content) | | |

3. Data represent the mean of difference in ear thickness (mm) ± S.T.D. (n = 12)
4. Control: Experimental group without drug treatment after inducing edema

As shown in the above Table 2, the Trachelospermi caulis extract of the present invention showed an excellent inhibitory effect against the arachidonic acid-induced ear edema. In particular, the highest edema inhibiting rate was observed when the concentration of the Trachelospermi caulis extract used was 400 mg/kg.

### Experimental Example 3

### Acetic Acid-Induced Writhing Response in Mice

The effect of Trachelospermi caulis extract obtained in Example 1 on acetic acid-induced writhing response in mice was investigated. The result is shown in the following Table 3.

[Test Method 3]

Twenty-four hour fasted 7 week old ICR mice were separated into each experimental group, based on the “Siegmund” method, orally administered with Joins® (SK Pharma Co., Ltd., Korea) at a concentration of 400 mg/kg and 200 mg/kg, and Trachelospermi caulis extract at a concentration of 400 mg/kg, 200 mg/kg and 20 mg/kg, respectively. One hour after the oral administration, each mouse was intraperitoneally administered with acetic acid (0.7%) at a concentration of 0.1 mL/10 g thereby inducing intestinal edema. Ten minutes after the intraperitoneal administration, each mouse was observed by means of stretching and the result was used as algesia index.

TABLE 3

| | Writhing | Analgesic |
|---|---|---|
| Treatment | No. ± S.D. | Rate (%) |
| Negative Control | 34.65 ± 1.6 | — |
| Positive Control (SK- Joins ®) | 17.5 ± 1.92 | 49 |
| 400 mg/kg | | |
| Positive Control (SK- Joins ®) | 19.9 ± 1.39 | 43 |
| 200 mg/kg | | |
| Trachelospermi caulis (400 mg/kg - | 18.8 ± 1.58 | 46 |
| 4.0 wt % of arctiin content) | | |
| Trachelospermi caulis (200 mg/kg - | 20.5 ± 1.3 | 41 |
| 4.0 wt % of arctiin content) | | |
| Trachelospermi caulis (20 mg/kg - | 32.3 ± 1.98 | 7 |
| 4.0 wt % of arctiin content) | | |

5. Data represent the mean of difference in writhing number (mm) ± S.T.D. (n = 12)
6. Control: Experimental group without drug treatment after inducing stretching

As shown in the above Table 3, the Trachelospermi caulis extract of the present invention showed an excellent inhibitory effect against the acetic acid-induced stretching. In particular, the greatest inhibitory effect against the acetic acid-induced analgesic effect was observed when the concentration of the Trachelospermi caulis extract used was 400 mg/kg.

### Experimental Example 4

### Assay of the Effects of Trachelospermi caulis Extract for the Treatment of Acute Arthritis in SD-Rats

The effect of Trachelospermi caulis extract obtained in Example 1 to treat acute arthritis was investigated. The result is shown in the following Table 4.

[Test Method 4]

Twenty-four hour fasted male SD rats with about 200 g of body weight were separated into each experimental group, and then orally administered with Joins® (SK Pharma Co., Ltd., Korea) at a concentration of 400 mg/kg and 200 mg/kg, and Trachelospermi caulis extract at a concentration of 400 mg/kg, 200 mg/kg and 100 mg/kg, respectively, and then injected on the left plantar with 100 μL of 1% carageenan physiological saline solution thereby inducing acute paw edema. Three hours later, thus induced edema was analyzed by measuring the volume of the edema using plethysmometer LE 7500.

TABLE 4

| Treatment | Inflammation Increase Rate (%) |
|---|---|
| Negative Control | 89 ± 1.67 |
| Positive Control (SK- Joins ®) | 43 ± 3.9 |
| 400 mg/kg | |
| Positive Control (SK- Joins ®) | 47 ± 3.45 |
| 200 mg/kg | |
| Trachelospermi caulis (400 mg/kg - | 45 ± 4.31 |
| 4.0 wt % of arctiin content) | |
| Trachelospermi caulis (200 mg/kg - | 49 ± 2.92 |
| 4.0 wt % of arctiin content) | |
| Trachelospermi caulis (100 mg/kg - | 59 ± 1.32 |
| 4.0 wt % of arctiin content) | |

7. Data represent the mean of difference in paw edema (mL) ± S.E.M. (n = 12)
8. Control: Experimental group without drug treatment after inducing edema

As shown in the above Table 4, the Trachelospermi caulis extract of the present invention showed an excellent therapeutic effect for the treatment of acute arthritis. In particular, the highest therapeutic effect was observed when the concentration of the Trachelospermi caulis extract used was 400 mg/kg.

### Experimental Example 5

### Assay of the Effects of Trachelospermi caulis Extract on the Production of No, iNOS and TNF-α

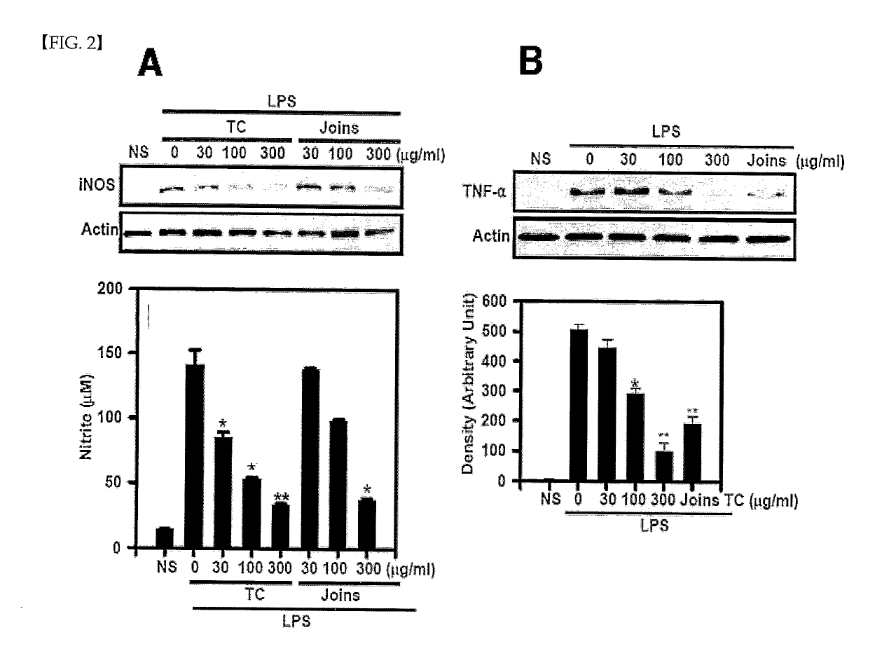

The effect of Trachelospermi caulis extract obtained in Example 1 to treat factors associated with inflammation was investigated. The result is shown in FIG. 2.

[Test Method]

Raw 264.7 cell line, a macrophage derived from a mouse, was treated with LPS (1 μg/mL). Twenty four hours later, the culture medium was collected, analyzed by using Griess reagent for nitric oxide (NO) assay. Four hours later, the cells of the resultant were lysed, and the effects of the Trachelospermi caulis extract on the expression level of inducible nitric oxide synthase (iNOS), and TNF-α, which are inflammation-inducing factors in the body, were measured via Western blot using iNOS or TNF-α antibodies.

As for the nitric oxide, 100 μL of the 24 hour culture medium was combined with 100 μL of the Griess reagent and its optical density was measured at OD 540 nm.

As for the iNOS and TNF-α, the raw 264.7 cell line was treated with LPS (1 μg/mL). Four hours later, the cells of the resultant were lysed and the whole cell lysate proteins were collected. The proteins were then separated by electrophoresis and then transferred to a nylon membrane for Western blot, and the amount of the proteins were analyzed by using iNOS and TNF-α antibodies.

As shown in FIG. 2, the Trachelospermi caulis extract exhibited an inhibitory effect against the expression of iNOS and TNF-α, as well as the production of NO by LPS thereby showing its excellent anti-inflammatory effect.

### Experimental Example 6

### Effects of Trachelospermi caulis Extract on Activation of MAP Kinase and NF κB in Macrophages

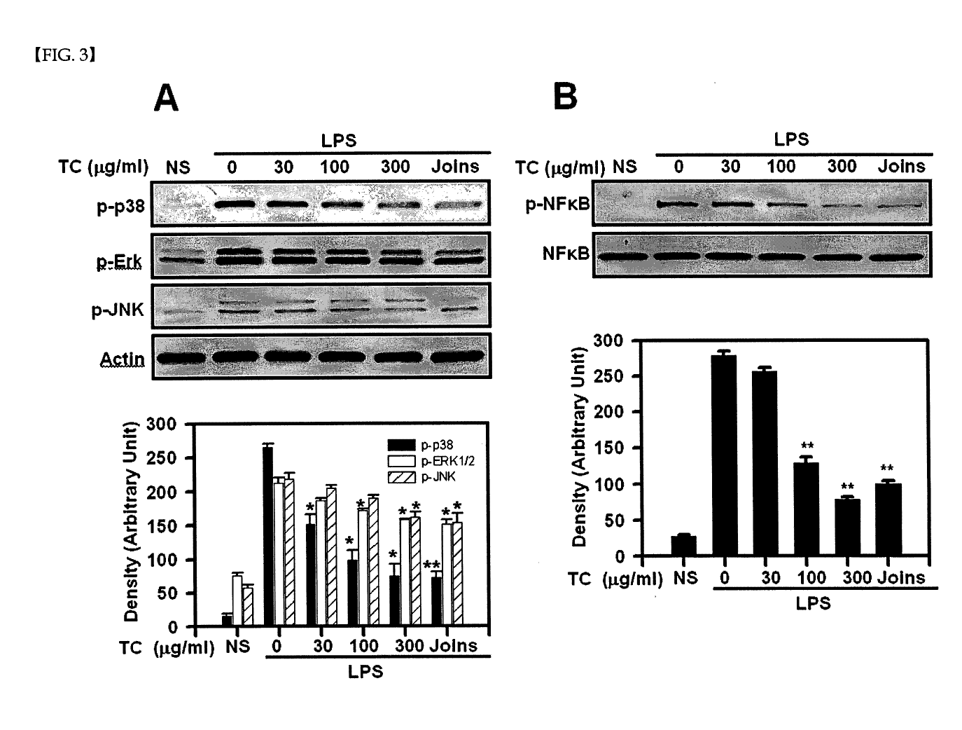

The effect of Trachelospermi caulis extract obtained in Example 1 to inhibit factors associated with inflammation on the activation of MAP kinases and NF κB were analyzed. The result is shown in FIG. 3.

[Test Method] Western Blot Analysis

LPS cells treated along with the Trachelospermi caulis extract were washed with PBS and then dissolved in lysis buffer for 30 minutes. The resulting lysate was centrifuged at 4° C. at the rate of 12,000 rpm for 15 minutes. The supernatant was dissolved twice in Laemmli buffer, separated by SDS-PAGE gel and then transferred to a nitrocellulose membrane.

Examples of the first antibody are anti-iNOS, anti-phosph-p38, anti-phosph-NF-κB or other antibodies.

Examples of the second antibody are those indicated as horseradish peroxidase (HRP).

As shown in FIG. 3, the Trachelospermi caulis extract was shown to inhibit the activation of p38 MAP kinase and NF κB.

### Experimental Example 7

### Test of Acute Toxicity

One gram of the Trachelospermi caulis extract obtained in Example 1 was used to identify the presence of any toxicity that may be resulted from repetitive administration of 1 g of the Trachelospermi caulis extract. Sixteen hour-fasted 4-5 week old ICR mice (7 mice per group) were selected as experimental animals.

One gram of the Trachelospermi caulis extract dissolved in 0.5% carboxymethylcellulose (CMC) oral administration of was repeatedly administered orally for a period of 5 days and none of the mice were found dead and also no damages on internal organs were noticed.

### Preparation Example 1

### Manufacture of Tablets

Tablets for oral administration with the composition as set forth below comprising the Trachelospermi caulis extract were manufactured via wet granulation and dry granulation methods.

### [Composition]

| | | |
|---|---|---|
| Trachelospermi caulis extract | 200 | mg |
| light anhydrous silicic acid | 10 | mg |
| magnesium stearate | 2 | mg |
| microcrystalline cellulose | 50 | mg |
| sodium starch glycolate | 25 | mg |
| povidone | 12 | mg |

| | |
|---|---|
| anhydrous ethanol | adequate |

### Preparation Example 2

### Manufacture of Ointments

Ointments with the composition as set forth below comprising the Trachelospermi caulis extract were manufactured.

### [Composition]

| | | |
|---|---|---|
| Trachelospermi caulis extract | 5 | g |
| cetyl palmitate | 20 | g |
| cetanol | 40 | g |
| stearyl alcohol | 40 | g |
| isopropyl myristate | 80 | g |
| sorbitan monostearate | 20 | g |
| polysorbate | 60 | g |
| propyl p-oxybenzoate | 1 | g |
| methyl p-oxybenzoate | 1 | g |

| | |
|---|---|
| phosphoric acid and sterile water | adequate |

### Preparation Example 3

### Manufacture of Injections

Injections with the composition as set forth below comprising the Trachelospermi caulis extract were manufactured.

### [Composition]

| | | |
|---|---|---|
| Trachelospermi caulis extract | 100 | mg |
| mannitol | 180 | mg |
| sodium phosphate dibasic | 25 | mg |
| sterile water for injection | 2974 | mg |

### Preparation Example 4

### Manufacture of Transdermal Agents

Transdermal agents with the composition as set forth below comprising the Trachelospermi caulis extract were manufactured.

### [Composition]

| | | |
|---|---|---|
| Trachelospermi caulis extract | 100 | mg |
| sodium polyacrylate | 1.3 | g |
| glycerin | 3.6 | g |
| aluminum hydroxide | 0.004 | g |
| methyl parabene | 0.2 | g |
| acrylic adhesive solution | 14 | mL |

As described above, the Trachelospermi caulis extract of the present invention exhibits excellent inhibitory effects on pains resulted from acute inflammation, production of NO and TNF-α, activation of p38 MAP kinase and NF κB and acute edema, and thus by adjusting the components and their respective amounts effective in the prevention and treatment of inflammatory diseases the standardization of the Trachelospermi caulis extract can be attained.

Further, the Trachelospermi caulis extract of the present invention is extracted from Trachelospermi caulis, where arctiin is indicated as a biomarker, and by adjusting the amount of the biomarker it is possible to manufacture a therapeutic agent for the treatment of arthritis.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

1. A pharmaceutical composition comprising Trachelospermi caulis extracts for prevention and treatment of inflammatory diseases.

2. The pharmaceutical composition as claimed in claim 1, wherein the inflammatory disease is selected from arthritis and edema.

3. A method for preparing Trachelospermi caulis extract comprising:
(a) extracting Trachelospermi caulis with a solvent selected from the group consisting of water, alcohol and an aqueous alcohol solution and filtering the extract;
(b) separating aqueous layers from the resulting filtrate; and
(c) concentrating the aqueous layers to obtain a soluble solid.

4. The method as claimed in claim 3, wherein the concentrating step (c) is performed under reduced pressure.

5. The method as claimed in claim 3, which further comprises:
(d) suspending the resulting concentrate by using an alcohol and filtrating the resulting suspension by centrifugation;
(e) re-concentrating the resulting filtrate under reduced pressure; and
(f) drying, pulverizing and sterilizing the resulting concentrate.

6. The method as claimed in claim 4, which further comprises:
(d) suspending the resulting concentrate by using an alcohol and filtrating the resulting suspension by centrifugation;
(e) re-concentrating the resulting filtrate under reduced pressure; and
(f) drying, pulverizing and sterilizing the resulting concentrate.
